(19) United States
(12) Patent Application Publication
(10) Pub. No.: US 2024/0108469 A1
(43) Pub. Date: Apr. 4, 2024

(54) Extra Articular Stabilization Implant, Instrumentation, and Method

(71) Applicant: Oluwatodimu Richard Raji, San Francisco, CA (US)

(72) Inventor: Oluwatodimu Richard Raji, San Francisco, CA (US)

(21) Appl. No.: 18/476,979

(22) Filed: Sep. 28, 2023

Related U.S. Application Data
(60) Provisional application No. 63/377,660, filed on Sep. 29, 2022.

(51) Int. Cl. A61F 2/30

(52) U.S. Cl. CPC A61F 2/30988; A61F 2002/30149; A61F 2002/30289; A61F 2002/30327; A61F 2002/30622; A61F 2002/30851; A61F 2002/30995

(57) ABSTRACT

An extra-articular stabilization implant is disclosed. The extra-articular stabilization implant comprises: a body portion defining a longitudinal axis and having a proximal end and a distal end; a first helical thread formed on the body portion, the first helical thread having a first major diameter and a first minor diameter; a second helical thread formed on the body portion, the second helical thread having a second major diameter and a second minor diameter; and a head portion disposed at the proximal end of the body portion, the head portion having a driver receiving portion.

### FIELD OF THE INVENTION

The present invention relates generally to a method. More specifically, the present invention is a method for stabilization and fusing the sacroiliac joint.

### BACKGROUND OF THE INVENTION

The present invention is an improved method of coupling and stabilization and tools for accomplishing the same is disclosed.

### SUMMARY OF THE INVENTION

An extra-articular stabilization implant is disclosed. The extra-articular stabilization implant comprises: a body portion defining a longitudinal axis and having a proximal end and a distal end; a first helical thread formed on the body portion, the first helical thread having a first major diameter and a first minor diameter; a second helical thread formed on the body portion, the second helical thread having a second major diameter and a second minor diameter; and a head portion disposed at the proximal end of the body portion, the head portion having a driver receiving portion.

In one embodiment, the body portion is a tube defined by an inner surface of the first helical thread and the second helical thread.

In some embodiments, the driver receiving portion includes a pentagonal opening.

In some other embodiments, the driver receiving portion includes a radial notch.

### BRIEF DESCRIPTION OF THE DRAWINGS

FIG. 1 is an illustration of one embodiment of the present invention.

FIG. 2 is a side view of one embodiment of the present invention.

FIG. 3 is an illustration of one embodiment of conceptual placement of the present invention.

FIG. 4 is a top view of one embodiment of the present invention.

### DETAIL DESCRIPTIONS OF THE INVENTION

All illustrations of the drawings are for the purpose of describing selected versions of the present invention and are not intended to limit the scope of the present invention.

### Overview

An improved method and device for coupling and stabilization and tools for accomplishing the same is disclosed. In one embodiment, the present invention is a method that uses an extra-articular stabilization device that includes creating a single incision in the patient's skin proximal to the patient's sacroiliac joint, driving a stabilization implant having at least one coupling element passing through the ilium, across the sacroiliac joint, and into the sacrum, and vice versa, to embed into the bone tissue, and intertwine around the articular surfaces of the sacrum and the ilium, for form stabilization, while exerting axial, tangential and radial forces to compress or distract the joint surfaces, for force stabilization of the sacroiliac joint, without requiring transecting of the existing ligaments. This invention is applicable for the stabilization of biological and synthetic fragments and joints, such as the pubic symphysis, intervertebral disc space, facet joints, etc.

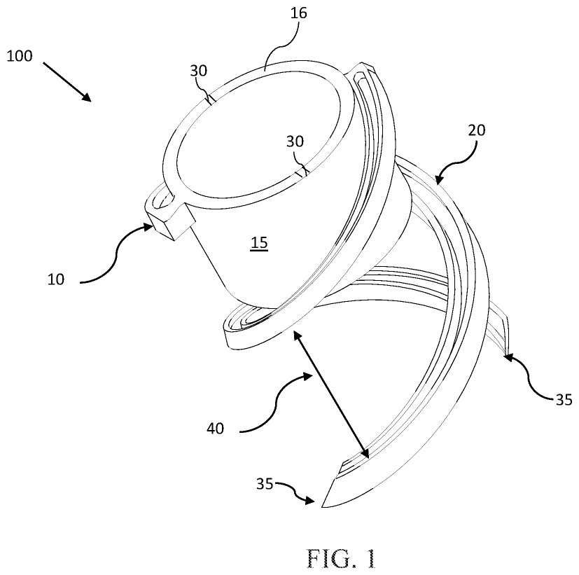

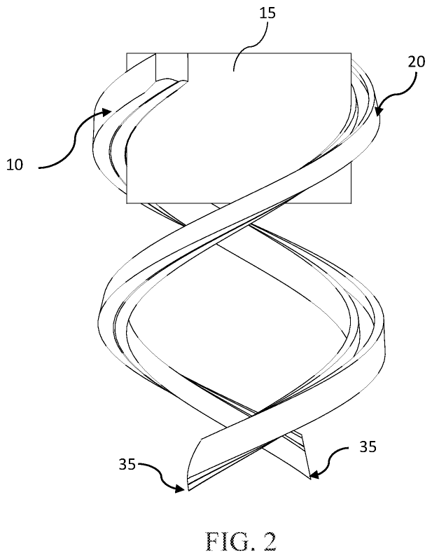

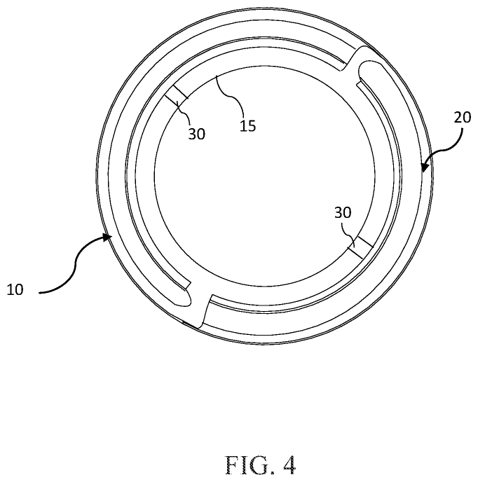

FIGS. 1-4, display a perspective view of the extra-articular stabilization implant 100. The implant 100 may include one or more helical elements 10, 20 extending from the implant body 15. Two helical elements (threads) 10, 20 in a single body are displayed, but other embodiments may have anywhere from one to ten helices which may be assembled and disassembled as separate bodies. The helical elements (threads) 10, 20 shown may have similar or different diameters, profiles, directions of twist, or porosities. The profiles of the helical elements 10, 20 shown may include rectangular and may also be circular, elliptical, polygonal, or triangular with chamfered or fileted edges to relieve stress on the adjacent bone. The implant body 100 may be solid or porous, with a driving feature.

In one embodiment, the implant body 100 of the present invention may include a slot (driver receiving portion) 30 but the driving feature may be female hexagonal, male, hexalobe, square, or cross in other embodiments. In some embodiments, the implant body 100 may include distal cutting and/or piercing features, which concurrently removes and/or recesses the tissue and embeds the implant body 100, without requiring the creation of a void beforehand. The implant body 100 and helical elements (threads) 10, 20 may be solid or perforated with hole(s) to allow for bony ingrowth through the implant 100.

In one embodiment, the implant body 100 is cannulated with a central lumen to allow for guided delivery and to allow for insertion of orthobiologic material before/after placement in the joint. This cannulation may be threaded to help retain the implant 100 to insertion instruments.

In some embodiments, the present invention may include sharp distal elements 35 (at the bottom of the helical elements (threads) 10, 20) to aid concurrent self-tapping and self-cutting into the sacral and iliac bones. These features 10,20 may also help to interlock the bone/material upon embedment. The sharp distal elements 35 may include features having a tapered curvilinear or triangular profile with either relief cut(s) or cutting flute(s), which may or may not aim distally and/or be aligned to the curvature of the helical elements (threads) 10, 20.

In some embodiments, the sharp distal elements 35 may include channels along its external, internal, superior or inferior surfaces to enable material transport during insertion. However, other embodiments may not include these sharp distal elements, wherein the Helical elements may be embedded into pre-drilled or pre-tapped helical channels of similar or different profiles.

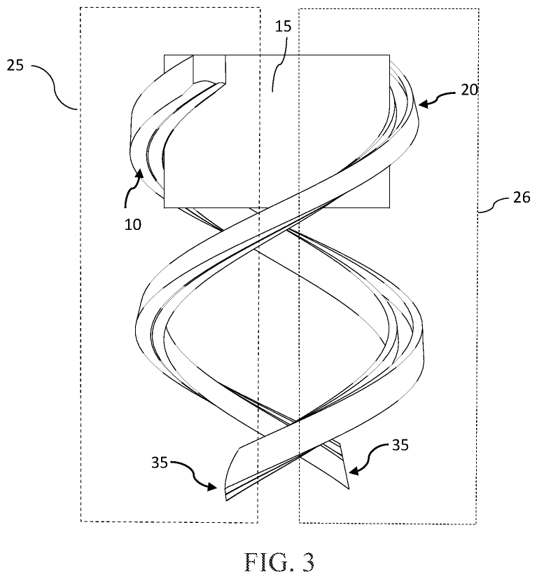

The FIG. 3 displays the conceptual placement of the extra-articular implant. Two blocks 25, 26 are displayed to represent the sacral and iliac bones. The right block 26 represents the soft cancellous bone and the left block 25 represents the hard cortical bone.

The implant 100 is placed flush against the posterior surface of the joint. The implant body 15, helical elements 10, 20, and sharp distal elements 35 are embedded into the soft cancellous (right block 26) and hard cortical bones (left block 25) without the creation of a void or the transecting of ligaments, for form closure stabilization. The starting position of the implant 100 relative to the bone structure determines the force closure stabilization mechanism. If the tips of the implant 100 are aligned to begin embedding when aligned collinear with the joint line, the tangential forces shall concurrently push the joint surfaces apart and embed, resulting in the maintained distraction of the joint and ligament axis at the insertion site, by radial forces.

Inversely, if the tips of the implant 100 are aligned to begin embedding when aligned perpendicular with the joint line, the tangential forces shall push the joint surfaces towards each other resulting in maintained compression of the joint surface at the insertion site by radial forces. In the same manner, if both surfaces or helixes 10, 20 are offset from each other, the axial force applied during insertion shall apply a shear force between the joint surfaces, to concurrently distract the ligaments which resist the shear motions during embedment.

In one embodiment, the extra-articular stabilization implant 100 may comprise a body portion 15 defining a longitudinal axis and having a proximal end and a distal end; a first helical thread 10 formed on the body portion 15, the first helical thread 10 having a first major diameter and a first minor diameter, the first helical thread 10 includes a sharp bottom end;

a second helical thread 20 formed on the body portion 15, the second helical thread 20 having a second major diameter and a second minor diameter, the second helical thread 20 includes a sharp bottom end; and
a head portion 16 disposed at the proximal end of the body portion, the head portion 16 having a driver receiving portion 30.

In some embodiments, the body portion 15 can be a tube defined by an inner surface of the first helical thread 10 and the second helical thread 20. The driver receiving portion 30 may include a pentagonal opening. In some embodiments, the driver receiving portion 30 may include a radial notch.

In some embodiments, the first major diameter and the second major diameter can be equal. The first minor diameter and the second minor diameter can also be equal.

In preferred embodiment, the present invention includes a gap 40 between the first helical thread 10 and the second helical thread 20.

Although the invention has been explained in relation to its preferred embodiment, it is to be understood that many other possible modifications and variations can be made without departing from the spirit and scope of the invention.

What is claimed is:

1. An extra-articular stabilization implant comprising:
a body portion defining a longitudinal axis and having a proximal end and a distal end;
a first helical thread formed on the body portion, the first helical thread having a first major diameter and a first minor diameter, the first helical thread includes a sharp bottom end, wherein the sharp bottom end and the helical thread are configured to be embedded into fragments to be coupled;
a second helical thread formed on the body portion, the second helical thread having a second major diameter and a second minor diameter, the second helical thread includes a sharp bottom end, wherein the sharp bottom end and the helical thread are configured to be embedded into fragments to be coupled; and
a head portion disposed at the proximal end of the body portion, the head portion having a driver receiving portion.

2. The extra-articular stabilization implant as claimed in claim 1, the body portion is a tube defined by an inner surface of the first helical thread and the second helical thread.

3. The extra-articular stabilization implant as claimed in claim 2, the driver receiving portion includes a pentagonal opening.

4. The extra-articular stabilization implant as claimed in claim 2, the driver receiving portion includes a radial notch.

5. The extra-articular stabilization implant as claimed in claim 4, the first major diameter and the second major diameter are equal.

6. The extra-articular stabilization implant as claimed in claim 4, the first minor diameter and the second minor diameter are equal.

7. The extra-articular stabilization implant as claimed in claim 6, wherein the extra-articular stabilization implant further comprises a gap between the first helical thread and the second helical thread.

8. An extra-articular stabilization implant comprising:
a body portion defining a longitudinal axis and having a proximal end and a distal end, the body portion is a tube defined by an inner surface of the first helical thread and the second helical thread;
a first helical thread formed on the body portion, the first helical thread having a first major diameter and a first minor diameter;
a second helical thread formed on the body portion, the second helical thread having a second major diameter and a second minor diameter; and
a head portion disposed at the proximal end of the body portion, the head portion having a driver receiving portion.

9. The extra-articular stabilization implant as claimed in claim 8, the driver receiving portion includes a pentagonal opening.

10. The extra-articular stabilization implant as claimed in claim 9, the driver receiving portion includes a radial notch.

11. The extra-articular stabilization implant as claimed in claim 10, the first major diameter and the second major diameter are equal.

12. The extra-articular stabilization implant as claimed in claim 10, the first minor diameter and the second minor diameter are equal.

13. The extra-articular stabilization implant as claimed in claim 12, wherein the extra-articular stabilization implant further comprises a gap between the first helical thread and the second helical thread.

14. An extra-articular stabilization implant comprising:
a body portion defining a longitudinal axis and having a proximal end and a distal end;
a first helical thread formed on the body portion, the first helical thread having a first major diameter and a first minor diameter;
a second helical thread formed on the body portion, the second helical thread having a second major diameter and a second minor diameter; and
a head portion disposed at the proximal end of the body portion, the head portion having a driver receiving portion which includes a pentagonal opening.

15. The extra-articular stabilization implant as claimed in claim 14, the body portion is a tube defined by an inner surface of the first helical thread and the second helical thread.

16. The extra-articular stabilization implant as claimed in claim 15, the driver receiving portion includes a radial notch.

17. The extra-articular stabilization implant as claimed in claim 16, the first major diameter and the second major diameter are equal.

18. The extra-articular stabilization implant as claimed in claim 16, the first minor diameter and the second minor diameter are equal.

19. The extra-articular stabilization implant as claimed in claim 18, wherein the extra-articular stabilization implant further comprises a gap between the first helical thread and the second helical thread.
